(19) United States
(12) Patent Application Publication
(10) Pub. No.: US 2014/0303497 A1
(43) Pub. Date: Oct. 9, 2014

(54) VISUALISATION CATHETER

(71) Applicant: Miles Charles Deholm DALBY, Buckinghamshire (GB)

(72) Inventor: Miles Charles Deholm Dalby, Buckinghamshire (GB)

(21) Appl. No.: 14/352,648

(22) PCT Filed: Oct. 17, 2012
(86) PCT No.: PCT/GB2012/052559

§ 371 Date: Apr. 17, 2014

(30) Foreign Application Priority Data
Oct. 17, 2011 (GB) 1117891.0

(51) Int. Cl. A61B 6/00

(52) U.S. Cl. CPC A61B 6/503; A61B 6/481
USPC 600/435

(57) ABSTRACT

The present invention relates to a visualisation device for visualising a spatial orientation of a feature, such as a constriction, inside a body lumen. In particular, the present invention relates to a visualisation device for visualising the spatial orientation of the aortic valve in the aorta for use during procedures such as, but not limited to, Transcatheter Aortic Valve Implantation (TAVI). The visualisation device comprises a distal portion that assumes a loop shape or form, extending in an axis that is transverse from the axis of the length of the device at least temporarily (e.g. when a guide wire is removed, the distal portion may assume the loop shape). In that distal portion is located a number of openings coupled to a first lumen for receiving a radiopaque material. The openings face away from the proximal end of the catheter (i.e. towards the feature to be imaged) in an axis that is generally parallel to the axis of the catheter. This configuration of openings enables a radiopaque material to be delivered to the desired site efficiently.

### FIELD OF THE INVENTION

The present invention relates to a visualisation device for visualising a spatial orientation of a feature, such as a constriction, inside a body lumen. In particular, the present invention relates to a visualisation device for visualising the spatial orientation of the aortic valve in the aorta for use during procedures such as, but not limited to, Transcatheter Aortic Valve Implantation (TAVI).

### BACKGROUND OF THE INVENTION

For interventional procedures, such as Transcatheter Aortic Valve Implantation (TAVI), where an aortic valve stenosis (calcified or blocked valve) or aortic valve regurgitation (a leaking valve) is treated by implanting a replacement valve via a suitable delivery catheter, it is important that the operator is able to view detail and orientation of the feature such as the valve. Without proper imaging of the site, it may be difficult for the operator to deliver the replacement valve (in a stent) reliably to the correct location. For example, in TAVI, insufficiently clear imaging of the aortic valve may make it very difficult for the operator to determine the correct vector for the guide wire to take, or to deliver the stent to the correct depth within the aortic valve.

In known techniques, a pigtail catheter is delivered to region of the aortic valve via a guide wire, similar to that shown in FIG. 1, to image the desired feature. The pigtail catheter is used to deliver an injected radiopaque material to the site of the aortic valve so that the site can be imaged on suitable X-Ray equipment. Since the radiopaque material can affect the functioning of organs, it is desirable to keep the volume of radiopaque material delivered to the site of the aortic valve to a minimum.

In using the known pigtail catheter to deliver the injected radiopaque material, it has been found that suitably detailed imaging rarely can be obtained. For example, it is often to be found that the pigtail locates in one of the aortic valve leaflets. In such situations, a detailed image of the leaflet in which the pigtail resides can be obtained, but very little, if any, detail of the other two leaflets can be obtained.

We have therefore appreciated the need for an improved visualisation device for use during such procedures so that a feature, such as a constriction, inside a body lumen may be better imaged.

### SUMMARY OF THE INVENTION

The present invention therefore provides a visualisation device for visualising a spatial orientation of a feature inside a body lumen, comprising: a flexible catheter sleeve, a distal portion connected to the catheter sleeve and configured to at least temporarily assume a loop shape extending in a plane that is transverse to an axis of the catheter sleeve; a first lumen extending through the flexible catheter sleeve and the distal portion for accommodating a radiopaque material; a plurality of openings in the distal portion connected to the first lumen for ejecting radiopaque material received in the first lumen, wherein the plurality of openings are arranged along at least a portion of the length of the distal portion in a surface of the distal portion that faces away from the catheter sleeve in a direction that is generally parallel to the axis of the catheter sleeve.

The loop shape of the distal portion may extend to reach the inner surface of a body lumen, for example the aorta, when a guide wire is withdrawn from the distal portion. This loop shape ensures that a sufficient surface area of the feature, for example the aortic valve, is adjacent the distal portion. Furthermore, by providing a plurality of openings facing a direction that is away from the catheter sleeve and in a direction that is generally parallel to the axis of the catheter (i.e. facing towards the constriction), this ensures that radiopaque material injected into the first lumen (and therefore ejected by the plurality of openings) is delivered across a relatively large proportion of the feature.

It would also be understood that the loop shape of the distal portion enables the orientation of the feature to be visualised by the operator, since misalignment of the imaging apparatus with respect to the desired plane of viewing the feature will be shown as a partial loop having a greater thickness than that when viewed along the correct plane (i.e. transverse the feature) or an ellipse or ring, instead of a single link.

In a first embodiment, the first lumen is configured to receive a guide wire. In this embodiment, the catheter may thus be guided using the guide wire, which is then retracted so that the radiopaque material may be injected into the first lumen, and delivered to the feature via the plurality of openings.

In this first embodiment, the catheter sleeve also comprises a first fenestration in the distal portion, coupled to the first lumen and configured to receive a guide wire.

In a second embodiment, of the visualisation catheter, the catheter sleeve comprises a second lumen for accommodating a guide wire. In this embodiment, the first and second lumen may therefore be isolated from each other.

In the second embodiment, the catheter may comprise a first fenestration in the distal portion, coupled to the second lumen and configured to receive a guide wire. By coupling the first fenestration to the second lumen, radiopaque material may be delivered to the feature via the plurality of openings, as compared to the openings and the first fenestration in the first embodiment.

In either first or second embodiments, the catheter may comprise a plurality of radiopaque markers along a portion of the catheter sleeve. Such radiopaque markers enable the operator to visualise the position of the catheter in the body lumen, for example its placement in the aorta. Furthermore, if the markers are spaced-apart by a known distance, for example 1 cm apart, the markers may be used to make relative measurements of surrounding features.

In either embodiment, the catheter comprises a plurality of radiopaque markers along a portion of the distal portion. Such radiopaque markers enable the operator to visualise the orientation of the loop of the catheter distal portion. When seated on the constriction in the body lumen, this also enables the orientation of the feature to be visualised, since the catheter loop will align itself approximately with the boundary of the feature.

In embodiments, the distal portion is configured such that, when the distal portion has assumed the loop shape, the axis of the loop shape is offset from the axis of the catheter sleeve proximal the distal portion. Such an offset configuration means that the visualisation catheter is away from axes that might otherwise be used by other catheters, for example a catheter used during a TAVI procedure.

According to the present invention, there is also provided a visualisation device for visualising a spatial orientation of a feature inside a body lumen, comprising a probe, the probe comprising: a flexible probe sleeve; a distal portion connected to the probe sleeve and configured to at least temporarily assume a loop having a plurality of waved portions, the loop extending in a plane that is generally transverse to an axis of the probe sleeve, and each of the plurality of waved portions extending in a plane that is generally perpendicular to the transverse plane, wherein, in use, the visualisation device is located adjacent a feature having a spatial orientation to be imaged, and wherein at least a portion of the distal portion is configured to be radiopaque for visualisation.

In embodiments, the distal portion comprises a plurality of radiopaque markers. Preferably, each of the plurality of markers is aligned with a minimum in a trough of a waved portion.

In alternative embodiments, the distal portion comprises a radiopaque material.

In some embodiments, wherein the distal portion comprises a shaped wire.

In alternative embodiments, the probe comprises a catheter, and wherein the flexible probe sleeve comprises a flexible catheter sleeve. Preferably, the catheter comprises: a first lumen extending through the flexible catheter sleeve and the distal portion for accommodating a radiopaque material; and a plurality of openings in the distal portion connected to the first lumen for ejecting radiopaque material received in the first lumen.

In these embodiments, the first lumen is configured to receive a guide wire. Furthermore, the catheter sleeve comprises a first fenestration in the distal portion, coupled to the first lumen and configured to receive a guide wire.

In some embodiments, the catheter sleeve comprises a second lumen for accommodating a guide wire. In these embodiments, the catheter comprises a first fenestration in the distal portion, coupled to the second lumen and configured to receive a guide wire.

In further embodiments, the probe comprises a plurality of radiopaque markers along a portion of the probe sleeve.

In some embodiments, the distal portion is configured such that, when the distal portion has assumed the loop shape, the axis of the loop shape is offset from the axis of the probe sleeve proximal the distal portion.

In embodiments, the feature to be visualised is an aortic valve, and wherein each of the waved portions are shaped and dimensioned to conform to a respective leaflet of an aortic valve.

### LIST OF FIGURES

The present invention will now be described, by way of example only, and with reference to the accompanying figures, in which:

FIG. 1 shows in schematic sectional view an aorta with an aortic valve, with a guide wire being inserted therein;

FIG. 2 shows a schematic sectional view of the aorta of FIG. 1, with a catheter is inserted into the aorta via the guide wire;

FIG. 3 shows, in partial sectional view, an aortic valve with the visualisation catheter according to the present invention;

FIG. 4 shows the distal portion of the visualisation catheter when viewed from underneath (i.e. when viewed with the catheter sleeve stretching away from the viewer);

FIG. 5 shows a schematic view of a supplemental device for visualising a spatial orientation of a feature inside a body lumen;

FIG. 6 shows the device of FIG. 5 in a side view; and

FIG. 7 shows the device of FIGS. 5 and 6 when used to visualise the orientation of an aortic valve.

### DETAILED DESCRIPTION OF THE PREFERRED EMBODIMENTS

Whilst we describe the visualisation catheter of the present invention in relation to procedures such as TAVI, it would be appreciated by the skilled reader that the catheter is not limited to the TAVI procedure. The catheter may be used in a number of interventional procedures in which imaging of a feature in a body lumen is desirable.

In brief, the present invention provides a visualisation device comprising a catheter having a distal portion that assumes a loop shape or form, extending in an axis that is transverse from the axis of the length of the catheter at least temporarily (e.g. when a guide wire is removed, the distal portion may assume the loop shape). In that distal portion is located a number of openings, facing away from the proximal end of the catheter (i.e. towards the feature to be imaged) in an axis that is generally parallel to the axis of the catheter. This configuration of openings, which are coupled to a suitable lumen within the catheter, enable radiopaque material to be delivered to the desired site efficiently.

In a supplemental invention, we describe a visualisation device that has a distal portion having a loop portion extending from the sleeve and that has a plurality of waved portions. The loop extends transverse to the sleeve, and the wave portions extend generally perpendicular to the transverse plane of the loop. As such, the distal portion is shaped to sit inside a feature, for example the leaflets of an aortic valve, to be imaged. The distal portion can be made of a radiopaque material, or have radiopaque markers therein. Alternatively, the device could comprise a catheter having a shaped distal portion as described above, with openings to eject a radiopaque material into the feature.

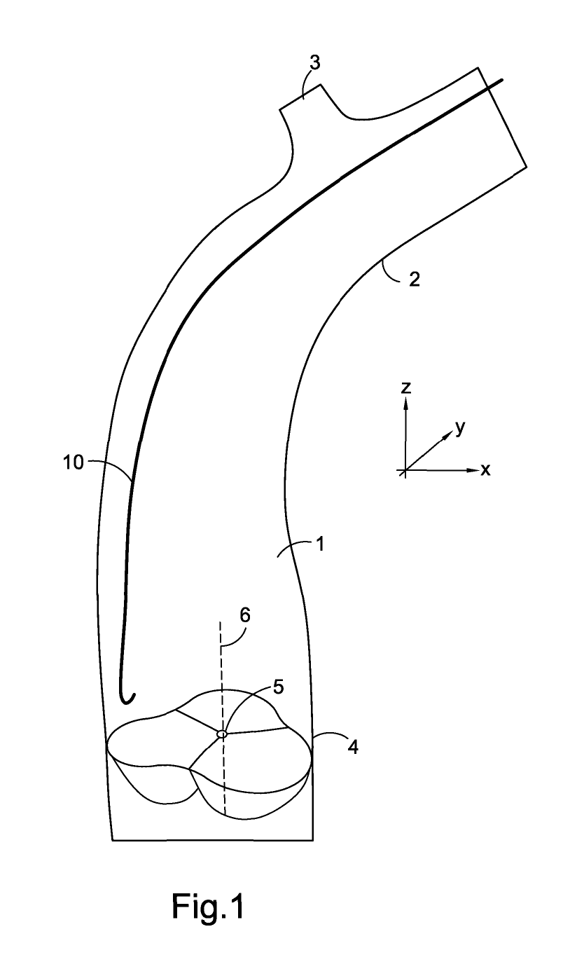

FIG. 1 (not drawn to scale), shows a perspective view of a guide wire 10 placed in an aorta 1 comprising aortic walls 2, a side vessel 3 as well as an aortic valve 4. The guide wire is delivered to the site using known techniques. In a procedure such as TAVI, the aortic valve 4 is the valve to be replaced, for example when the valve experiences stenosis (blocking) or regurgitation (leaking).

The coordinate system referred to in the figures indicates the distribution of spatial axes with respect to aortic valve 4, whereby XY is a plane parallel to the plane of aortic valve 4, whereas planes XZ and YZ are parallel to a longitudinal axis 6 of aorta 1. Also in the following figures coordinate systems indicate the plane corresponding to the shown perspective.

The aortic valve 4 has a residual opening 5, which is lying approximately centred to a longitudinal axis 6 of aorta 1. The longitudinal axis 6 is the optimal entry vector to the insertion of a stent (for example comprising a replacement valve) into the residual opening 5 when, for example, a calcified or leaking aortic valve is to be replaced in a TAVI procedure.

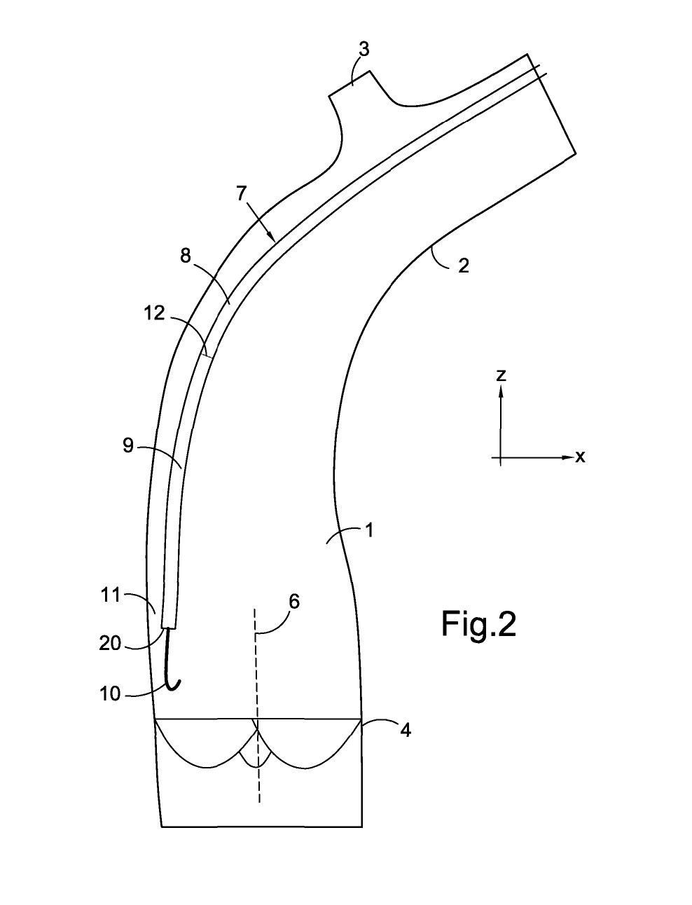

For imaging the desired feature (such as the aortic valve), a visualisation catheter according to the present invention is slid down the guide wire, as shown in FIG. 2 (also shown not to scale). The catheter 7 comprises a catheter sleeve 8 and a distal section 9, separated from the catheter sleeve by a proximal border 12. The catheter also comprises a first fenestration 20 at the distal end 11 of the catheter, through which the guide wire 10 exits the catheter.

Once the distal end 11 of the catheter has been located in the desired position, the guide wire 10 is retracted at least to the proximal border 12.

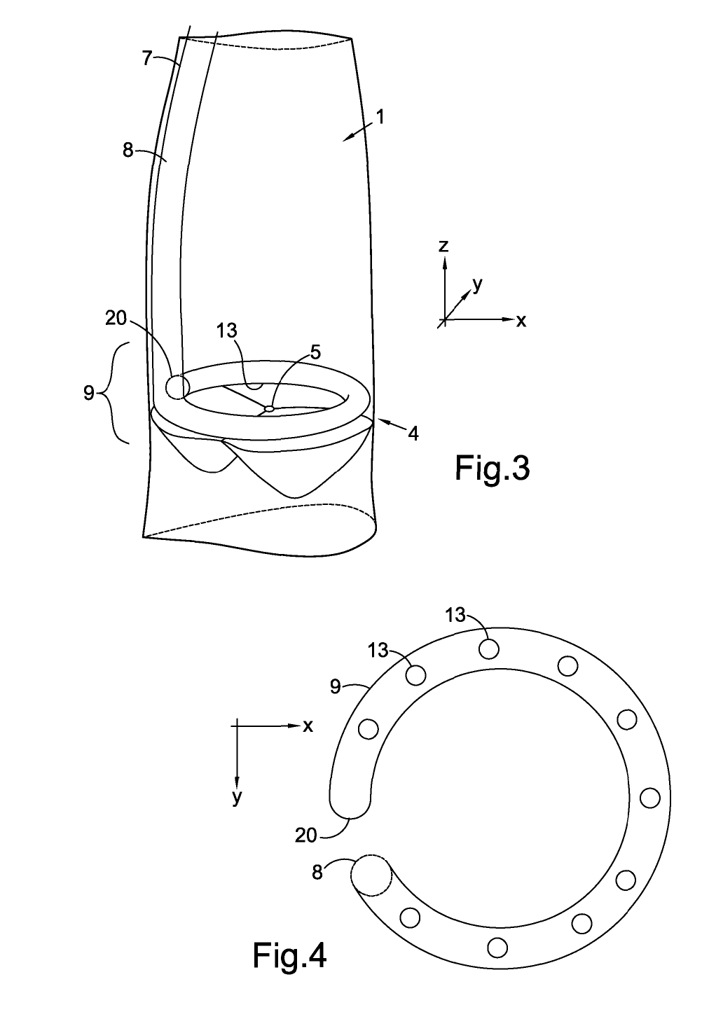

FIG. 3 (again, not shown to scale) shows the visualisation catheter 7 once the guide wire has been retracted beyond the proximal border. After retraction of the guide wire, the distal portion 9 assumes a generally loop shape or form, extending in an axis that is transverse to the axis of the catheter. Using the co-ordinates marked on the figures, if the catheter extends in the Z axis, the loop shape extends in the XY axis.

The loop-shaped distal portion 9 is configured to extend from the catheter 7 such that the axis of the loop is offset from the axis of the catheter 7. In this configuration, the catheter remains off the central axis of the aorta (or other body lumen if used in other procedures) so that other catheters may be placed in the body lumen for the procedure. Being loop-shaped means that the distal portion 9 therefore extends around the body lumen and at least partially covers each of the leaflets of the aortic valve (or other feature if used in other procedures).

The loop-shaped distal portion 9 further comprises a plurality of openings 13, through which radiopaque material may be ejected to enable the site of interest to be imaged on X-ray equipment. The openings are connected to a first lumen that runs through the catheter sleeve 8 and distal portion 9. The first lumen is configured to receive a radiopaque material for delivery, via the openings 13, to the site of interest.

FIG. 4 (not to scale) shows the ‘underside’ of the distal portion 9 (i.e. the catheter 7 extends away from the view). A plurality of openings 13 (coupled to the first lumen for receiving a radiopaque material) are located along at least part of the surface of the distal portion 9, directed away from the catheter, and generally in an axis that is parallel to the catheter. As such, when the distal portion 9 is located correctly in the aorta (or other body lumen), the radiopaque material will be ejected directly towards the constriction to be imaged.

Since the loop-shaped distal portion 9 extends around the periphery of the body lumen, and there are a plurality of openings 13 along the length of the distal portion 9, radiopaque material ejected from the openings 13 will substantially flow over the area of interest such that the feature may be imaged more reliably. For example in the aorta for imaging the aortic valve, the plurality of openings will means that sufficient radiopaque material will cover all three leaflets of the valve such that all of the leaflets may be visible using suitable X-ray imaging apparatus.

The distal portion 9 also comprises a fenestration 20, through which the guide wire may exit the distal end 11 of the catheter 7.

In a first embodiment, the fenestration 20 is connected to the first lumen (extending through the catheter sleeve 8 and distal section 9), along with the plurality of openings 13. In this embodiment, the single lumen therefore provides a simple visualisation catheter, since only one lumen is required for both guiding the visualisation catheter into position, and delivering the radiopaque material to the feature to be imaged (via the openings).

In this embodiment, the radiopaque material also will be ejected from the fenestration 20. Since the fenestration 20 needs to be large enough to receive the diameter of the guide wire 10, the openings 13 are dimensioned sufficiently large enough to encourage the radiopaque material to flow substantially equally out of the fenestration 20 and each of openings 13.

In a second embodiment, the fenestration 20 is connected to a second lumen, again extending through the catheter sleeve 8 and distal portion 9. The first and second lumens are not coupled together. As such, radiopaque material injected into the first lumen will only flow out of the opening 13. In this embodiment, the second lumen in primarily used for accommodating the guide wire 10 so that the visualisation catheter may be delivered to the site of interest.

However, in this second embodiment, the second lumen may also be used for remote monitoring of parameters (such as blood pressure) once the guide wire 10 has been removed.

Once in the loop shape, the catheter 7 and distal portion 9 is seated on, or in the region of, the aortic valve 4 (or other constriction) as required by the procedure.

The distal portion 9 may be manufactured from a radiopaque material so that the orientation of the distal portion 9 may be determined using X-ray imaging apparatus. Alternatively, the distal portion 9 may comprise a plurality of radiopaque markers, for example an annular structure around at least a portion of the loop. The structure may comprise alternate sections of long and short markers. Alternatively, the distal portion 9 may be configured to become visible by X-ray imaging when the distal portion 9 carries a radiopaque material to be delivered via the catheter to the site of interest.

Once in the loop shape, the distal portion 9 therefore provides an annular structure, which makes possible visualising of the position and tilt of aortic valve 4, as well as the catheter position itself.

When the X-ray imaging apparatus is in a position perpendicular to the plane of the aortic valve (for example in the orientation shown in FIG. 2), the distal portion 9 of the catheter 7 (if seated on or proximal the aortic valve) will show as a line. However, if the X-ray imaging apparatus is in a position that is off-axis relative to the perpendicular axis, the distal portion 9 will show as a thickened line, two lines separate by a gap, or an ellipse or a ring. As such, the operator can use the visualisation catheter to align the X-ray imaging apparatus more accurately.

Furthermore, the catheter sleeve 8 may be provided with a plurality of radiopaque markers along at least a portion of its length. Such markers may be used for determining a position or a path of the catheter in the body lumen when imaged using X-ray imaging apparatus. Furthermore, if the markers are spaced-apart by a known distance, for example 1 cm apart, the markers may be used to make relative measurements of surrounding features.

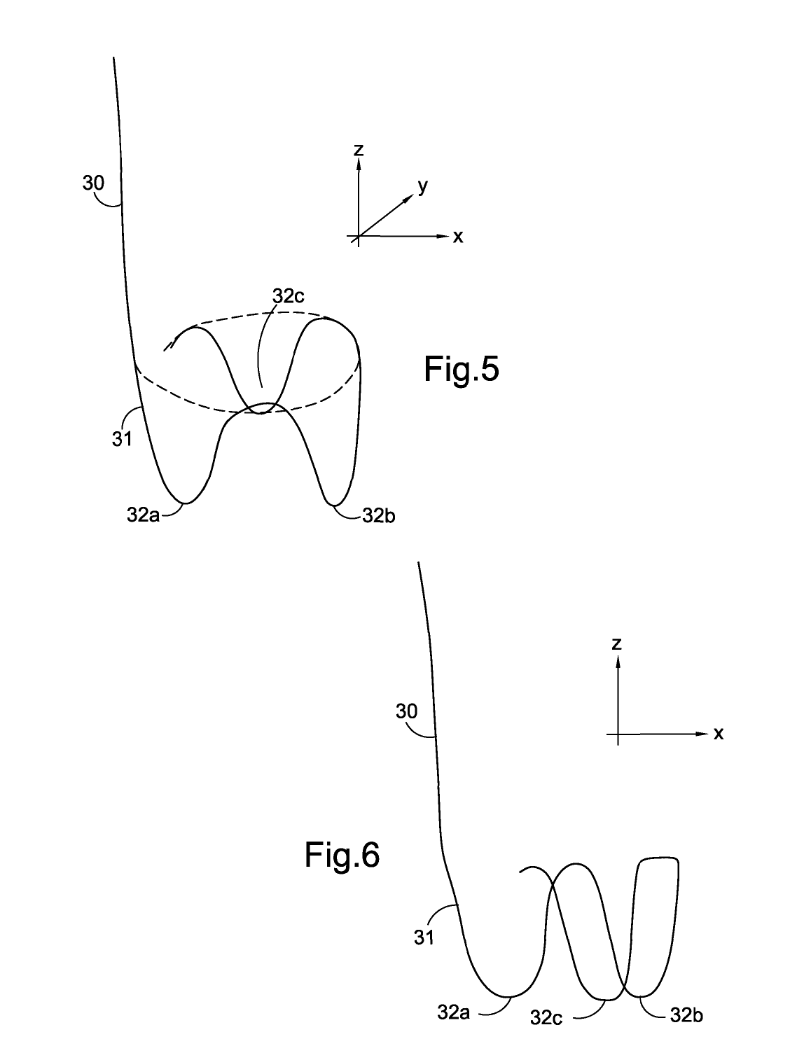

FIGS. 5 to 7 show a supplemental invention for the visualisation device. In this supplemental device, the distal portion is shaped to reside in the feature, for example the leaflets of the aortic valve 4.

In the supplemental invention, the visualisation device comprises a probe 30 having a distal portion 31, which is a loop extending generally in a plane that is transverse to the axis of the probe 30. The distal portion also comprises a plurality of waved features 32a, 32b, 32c, which extend from the transverse plane in a plane that is generally perpendicular to the transverse plane. In FIG. 5, the path the loop would take in the transverse plane without the waved portions is shown in dashed lines. FIG. 6 shows the probe in a side view.

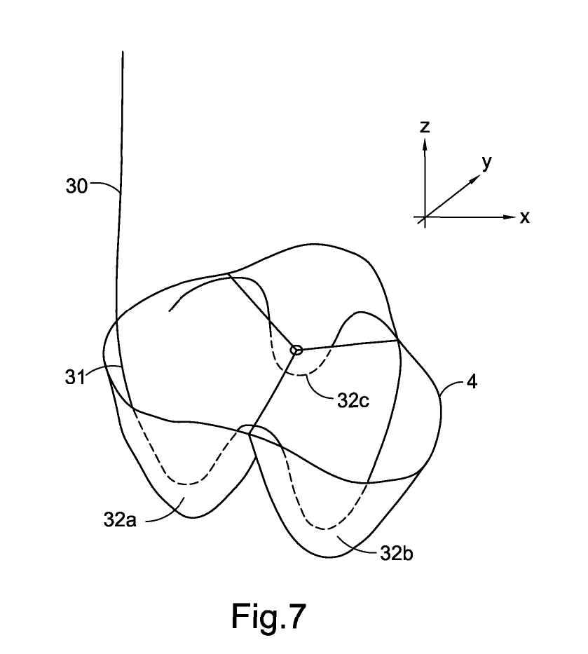

As can be seen in FIG. 7, the waved portions can be shaped so as to enable the distal portion to sit in the feature to be visualised (in this case, the leaflets of an aortic valve 4). By shaping the waved portions appropriately, the device may be placed in the aortic valve to enable the position and orientation of the valve to be visualised by an operator when using imaging apparatus, for example X-Ray imaging apparatus, as in the first invention.

In a first embodiment of the supplemental invention, at least the distal portion comprises a radiopaque material that is visible using X-Ray imaging apparatus.

In a second embodiment of the supplemental invention, the distal portion comprises a plurality of radiopaque markers to enable portions of the distal portion to be visualised using X-Ray imaging apparatus. For example, radiopaque markers may be placed a the minimum point of the waved portion, which would align approximately with the minimum point of the aortic leaflet. As such, the position and orientation of the leaflet can be imaged by the operator.

These first and second embodiments are advantageous in that the position and orientation of the feature may be visualised without the need for a fluid radiopaque material, which is toxic.

In a third embodiment of the supplemental invention, the probe comprises a catheter as with the first invention. That is, the catheter comprises a flexible catheter sleeve 30 and a distal portion 31. A first lumen, which is configured to accommodate a radiopaque material, extends through the catheter sleeve and shaped distal portion 31. The distal portion further comprises a plurality of openings, coupled to the first lumen, and configured to eject a radiopaque material that is injected into the first lumen. The distal portion further comprises a fenestration.

The first lumen is also configured to accommodate a guide wire, as in the first invention. The guide wire may exit the distal portion through the fenestration.

Once in place, the catheter will thus enable a radiopaque material to be injected into the leaflets of the aortic valve. Since the shape of the distal portion conforms to the shape of the leaflets, the radiopaque material is delivered to the desired site, and thus less radiopaque material may be required to visualise the area.

In a fourth embodiment, which is similar to the third embodiment, the catheter is provided with a second lumen. The first and second lumen are independent of one another, with the second lumen being configured to accommodate a guide wire, and the first lumen being configured to accommodate a radiopaque material. Only the first lumen is coupled to the openings. As with the first invention, the fenestration is coupled to the second lumen.

In either of the third or fourth embodiments, the distal portion may be made of a radiopaque material, or have radiopaque markers located along a portion, as with the first and second embodiments. Furthermore, since the distal portion in the third and fourth embodiments is carrying a radiopaque material, the distal portion may be configured to be radiopaque when carrying the material.

In any of the first to fourth embodiments of the supplemental invention, the probe sleeve may have radiopaque markers distributed along a portion to enable the position of the probe sleeve to be determined. If the distance between the markers is known, measurements may also be taken using these markers.

No doubt many other effective alternatives will occur to the skilled person. It will be understood that the invention is not limited to the described embodiments and encompasses modifications apparent to those skilled in the art lying within the scope of the claims appended hereto.

1. A visualisation device for visualising a spatial orientation of a feature inside a body lumen, comprising a catheter, the catheter comprising:
a flexible catheter sleeve,
a distal portion connected to the catheter sleeve and configured to at least temporarily assume a loop shape extending in a plane that is transverse to an axis of the catheter sleeve;
a first lumen extending through the flexible catheter sleeve and the distal portion for accommodating a radiopaque material;
a plurality of openings in the distal portion connected to the first lumen for ejecting radiopaque material received in the first lumen,
wherein the plurality of openings are arranged along at least a portion of the length of the distal portion in a surface of the distal portion that faces away from the catheter sleeve in a direction that is generally parallel to the axis of the catheter sleeve.

2. A visualisation device according to claim 1, wherein the first lumen is configured to receive a guide wire.

3. A visualisation device according to claim 2, wherein the catheter sleeve comprises a first fenestration in the distal portion, coupled to the first lumen and configured to receive a guide wire.

4. A visualisation device according to claim 1, wherein the catheter sleeve comprises a second lumen for accommodating a guide wire.

5. A visualisation device according to claim 4, wherein the catheter comprises a first fenestration in the distal portion, coupled to the second lumen and configured to receive a guide wire.

6. A visualisation device according to claim 1, wherein the catheter comprises a plurality of radiopaque markers along a portion of the catheter sleeve.

7. A visualisation device according to claim 1, wherein the catheter comprises a plurality of radiopaque markers along a portion of the distal portion.

8. A visualisation device according to claim 1, wherein the distal portion is configured such that, when the distal portion has assumed the loop shape, the axis of the loop shape is offset from the axis of the catheter sleeve proximal the distal portion.

9. (canceled)

10. A visualisation device for visualising a spatial orientation of a feature inside a body lumen, comprising a probe, the probe comprising:
a flexible probe sleeve;
a distal portion connected to the probe sleeve and configured to at least temporarily assume a loop having a plurality of waved portions, the loop extending in a plane that is generally transverse to an axis of the probe sleeve, and each of the plurality of waved portions extending in a plane that is generally perpendicular to the transverse plane,
wherein, in use, the visualisation device is located adjacent a feature having a spatial orientation to be imaged, and
wherein at least a portion of the distal portion is configured to be radiopaque for visualisation.

11. A visualisation device according to claim 10, wherein the distal portion comprises a plurality of radiopaque markers.

12. A visualisation device according to claim 11, wherein each of the plurality of markers is aligned with a minimum in a trough of a waved portion.

13. A visualisation device according to claim 10, wherein the distal portion comprises a radiopaque material.

14. A visualisation device according to claim 10, wherein the distal portion comprises a shaped wire.

15. A visualisation device according to claim 10, wherein the probe comprises a catheter, and wherein the flexible probe sleeve comprises a flexible catheter sleeve.

16. A visualisation device according to claim 15, wherein the catheter comprises:
a first lumen extending through the flexible catheter sleeve and the distal portion for accommodating a radiopaque material; and
a plurality of openings in the distal portion connected to the first lumen for ejecting radiopaque material received in the first lumen.

17. A visualisation device according to claim 16, wherein the first lumen is configured to receive a guide wire.

18. A visualisation device according to claim 17, wherein the catheter sleeve comprises a first fenestration in the distal portion, coupled to the first lumen and configured to receive a guide wire.

19. A visualisation device according to claim 16, wherein the catheter sleeve comprises a second lumen for accommodating a guide wire.

20. A visualisation device according to claim 19, wherein the catheter comprises a first fenestration in the distal portion, coupled to the second lumen and configured to receive a guide wire.

21. A visualisation device according to claim 10, wherein the probe comprises a plurality of radiopaque markers along a portion of the probe sleeve.

22. A visualisation device according to claim 10, wherein the distal portion is configured such that, when the distal portion has assumed the loop shape, the axis of the loop shape is offset from the axis of the probe sleeve proximal the distal portion.

23. A visualisation device according to claim 10, wherein the feature to be visualised is an aortic valve, and wherein each of the waved portions are shaped and dimensioned to conform to a respective leaflet of an aortic valve.
